(19) United States
(12) Patent Application Publication
(10) Pub. No.: US 2016/0045320 A1
(43) Pub. Date: Feb. 18, 2016

(54) VARIABLE HIP RESURFACING FEMORAL IMPLANT

(71) Applicant: RevOrtho LLC, Great Neck, NY (US)

(72) Inventors: Craig Eric Klinger, Great Neck, NY (US); Jose Bernardo Toro Arbelaez, Remsenburg-Speonk, NY (US)

(21) Appl. No.: 14/829,082

(22) Filed: Aug. 18, 2015

Related U.S. Application Data
(60) Provisional application No. 62/038,763, filed on Aug. 18, 2014.

(51) Int. Cl. A61F 2/36; A61B 17/16

(52) U.S. Cl. CPC A61F 2/3609; A61B 17/1668; A61F 2002/365

(57) ABSTRACT

A variable hip resurfacing implant which allows for varying configuration options to account for characteristics of underlying degenerative hip joint disease and for both un-cemented and cemented application. The variable hip resurfacing implant generally includes a femoral head portion, bearing surface, outer rim, inner planar surface, central stem, and a central stem threaded hole.

### CROSS-REFERENCE TO RELATED APPLICATIONS

The present application claims the benefit of U.S. Provisional Patent Application Ser. No. 62/038,763, filed Aug. 18, 2014, the entire contents of which is incorporated herein by reference thereto.

### FIELD OF THE INVENTION

The present invention relates to hip resurfacing arthroplasty.

### BACKGROUND INFORMATION

Degenerative conditions of the hip joint results in pain and disability and affects a large segment of the population. Conditions which can lead to onset of hip disease include cartilage injury, osteoarthritis, avascular necrosis, and other hip joint pathologies such as mechanical abnormalities. Multiple operative treatment modalities and implants have been promoted over time with the aim of relieving pain and restoration of improved hip joint mechanics. Initial attempts at surgical treatment completely removed and replaced the femoral head with a metallic implant, which was connected to a load-bearing femoral stem. This represented an early form of Hip Replacement Arthroplasty, a widely utilized surgical treatment which has continued through present times.

Hip Resurfacing Arthroplasty conserves more native bone including both the femoral head and femoral neck in contrast to Hip Replacement Arthroplasty. In Hip Resurfacing Arthroplasty the femoral head surface is removed and replaced using a hip resurfacing implant. The acetabulum is also resurfaced through implantation of an acetabular cup component. A typical hip resurfacing femoral implant is illustrated as prior art in FIG. 1B.

Hip Resurfacing Arthoplasty has its origins in the 1960's with early implants developed by John Charnley. More modern hip resurfacing implants were developed by Harlan Amstutz first, as illustrated in U.S. Pat. No. 4,123,806, showing a non-stemmed femoral hip resurfacing implant, and then, in U.S. Pat. No. 6,156,069, showing a stemmed femoral hip resurfacing implant. A similar hip resurfacing system was developed by Derrick McMinn, an orthopedic surgeon based in the West Midlands, UK. This particular system is known as the Birmingham Hip Resurfacing (BHR) system and features a metal-on-metal design for its bearing surfaces.

A limitation of the Amstutz and BHR systems is a lack of modularity through threaded attachments for supplementing fixation of the implant to the underlying bone. In these hip resurfacing systems, bone cement is applied to secure the femoral implant to the underlying bone. While these implants include porous surfaces which can accommodate bone ingrowth, the implant is initially secured to the underlying bone only with cement. Bone cement is relied upon for initial fixation of the implant to the femur as adequate bone ingrowth often takes months to develop. No additional fixation options are available in instances where poor bone quality is encountered. Local areas of bone deficiency can often be encountered in patients indicated for hip resurfacing arthroplasty and the lack of modular fixation options is a significant limitation of these systems. Localized bone deficiencies can be encountered in these patients due to avascular necrosis, localized bone cysts and localized osteolysis. In the event deficient cancellous bone is encountered during surgery these implants do not allow for supplementation of fixation through addition of threaded attachments. As such, the Amstutz and BHR hip resurfacing systems provide limited flexibility in addressing bone deficiency encountered during surgery. In U.S. Pat. Pub. No. 2014/0316531 by the present inventors, a modular hip resurfacing system is shown which overcomes most of the limitations of the Amstutz and BHR hip resurfacing system. However, the modular hip resurfacing system includes a step-shaped design/configuration along the undersurface of the cap of the hip resurfacing femoral implant, which consists of a central rim, an outer planar surface, and an inner rim, and a corresponding stepped osteotomy is performed along the femoral head for a shape-fit with the implant. In addition, the inner rim of the cap portion surrounds the femoral head cortex remaining above the articular rim.

### SUMMARY

The variable hip resurfacing implant according to an example embodiment of the present invention includes a cap portion having an inner rim having a non-stepped profile disposed along a periphery of an underside of the cap portion, and a central stem disposed at the underside of the cap portion. The central stem includes threading at a base of the central stem to accommodate threaded attachments. According to an example embodiment of the present invention, the undersurface of the implant does not have a step-shaped design or configuration along its periphery. The outer planar surface is not present in the variable hip resurfacing implant, and a single rim along the periphery of the undersurface is included. This thereby simplifies the design and manufacturing of the femoral implant as the number of contours are reduced. The associated osteotomy along the femoral head is also not step-shaped in design or configuration.

According to an example embodiment of the present invention, the inner rim of the cap portion does not surround the femoral head cortex remaining above the articular rim. In an alternative embodiment, the inner rim of the cap portion does surround the femoral head cortex remaining above the articular rim.

The variable hip resurfacing system according to an example embodiment of the present invention has threading at the base of its stem for modular attachments. This allows for the addition of a central screw, which can be used in combination with a femoral plate to significantly increase construct strength, when required.

An additional advantage of the variable hip resurfacing implant according to an example embodiment of the present invention is additional threaded holes along the undersurface of the upper cap portion. These additional threaded holes allow for multiple scalable fixation options, which can be used to further supplement construct strength in the setting of localized bone deficiencies. If localized bone deficiencies are encountered during the procedure fixation can be supplemented in the variable hip resurfacing system through the threaded attachment points along the undersurface of the implant. This is possible through supplemental threaded attachments through the use of additional screws or pegs for additional fixation points. The additional threaded holes along the undersurface of the variable hip resurfacing implant in the present invention allow for varying implantation to address bone deficiencies encountered during surgery as compared to traditional hip resurfacing. These modular fixation options allow for scalable options to increase implant strength, and provide both durability and rotational stability during the lifecycle of the hip resurfacing implant.

### BRIEF DESCRIPTION OF THE DRAWINGS

FIG. 1A is a front view from anterior of a human proximal femur.

FIG. 1B is a front view illustrating a proximal femur following implantation of a hip resurfacing femoral implant with a solid joined stem, according to a previous device. The dashed line illustrates the required bone cuts for typical hip resurfacing femoral implants.

FIG. 1C is a front view of a human proximal femur illustrating reference lines and angles for surgical preparation for a variable hip resurfacing implant according to the present invention.

FIG. 1D is a front view of a proximal femur following bone cuts for a variable hip resurfacing implant according to the present invention.

FIG. 2A is a front view illustrating a variable hip resurfacing implant with a fixed central stem, in accordance with one exemplary embodiment of the present invention.

FIG. 2B is a cross-sectional left view of the variable hip resurfacing implant shown in FIG. 2A, as indicated by line 2B-2B in FIG. 2A.

FIG. 2C is a right view of the variable hip resurfacing implant shown in FIG. 2A.

FIG. 2D is a cross-sectional front view of the variable hip resurfacing implant shown in FIG. 2A, as indicated by line 2D-2D in FIG. 2C.

FIG. 2E is a bottom view of the variable hip resurfacing implant shown in FIG. 2A.

FIG. 2F is a bottom perspective view of the variable hip resurfacing implant shown in FIG. 2A.

FIG. 2G is an enlarged bottom perspective view of the variable hip resurfacing implant shown in FIG. 2A in detail, as indicated by the dashed circle around FIG. 2F.

FIG. 3A is an exploded bottom perspective view, following associated bone cuts, illustrating placement of a variable hip resurfacing implant, one-hole plate, and a central screw, in accordance with one exemplary modular configuration of the present invention.

FIG. 3B is a bottom perspective view following placement of the variable hip resurfacing implant, one-hole plate, and a central screw shown in FIG. 3A.

FIG. 3C is a cross-sectional right view, as indicated by line 3C-3C in FIG. 3B, following placement of the variable hip resurfacing implant, one-hole plate, and a central screw shown in FIG. 3A.

FIG. 4A is an exploded bottom perspective view, following associated bone cuts, illustrating placement of the variable hip resurfacing implant, three-hole plate, two outer screws, and a central screw, in accordance with another exemplary modular configuration of the present invention.

FIG. 4B is a bottom perspective view following placement of the variable hip resurfacing implant, three-hole plate, two outer screws, and a central screw shown in FIG. 4A.

FIG. 4C is a cross-sectional right view, as indicated by line 4C-4C in FIG. 4B, following placement of the variable hip resurfacing implant, three-hole plate, two outer screws, and a central screw shown in FIG. 4A.

FIG. 5A is an exploded bottom perspective view, following associated bone cuts, illustrating placement of the variable hip resurfacing implant, two outer pegs, one-hole plate and a central screw, in accordance with another exemplary modular configuration of the present invention.

FIG. 5B is a bottom perspective view following placement of the variable hip resurfacing implant, two outer pegs, one-hole plate, and a central screw shown in FIG. 5A.

FIG. 5C is a cross-sectional right view, as indicated by line 5C-5C in FIG. 5B, following placement of the variable hip resurfacing implant, two outer pegs, one-hole plate, and a central screw shown in FIG. 5A.

FIG. 6A is an exploded bottom perspective view, following associated bone cuts, illustrating placement of the variable hip resurfacing implant, two outer pegs, and a threaded central cap, in accordance with another exemplary modular configuration of the present invention.

FIG. 6B is a bottom perspective view following placement of the variable hip resurfacing implant, two outer pegs, and the threaded central cap shown in FIG. 6A.

FIG. 6C is a cross-sectional right view, as indicated by line 6C-6C in FIG. 6B, following placement of the variable hip resurfacing implant, two outer pegs, and the threaded central cap shown in FIG. 6A.

FIG. 7A is an exploded bottom perspective view, following associated bone cuts, illustrating placement of the variable hip resurfacing implant, three-hole plate, two outer screws, and a central screw, in accordance with another exemplary modular configuration of the present invention.

FIG. 7B is a bottom perspective view following placement of the variable hip resurfacing implant, three-hole plate, two outer screws, and a central screw shown in FIG. 7A.

FIG. 7C is a cross-sectional right view, as indicated by line 7C-7C in FIG. 7B, following placement of the variable hip resurfacing implant, three-hole plate, two outer screws, and a central screw shown in FIG. 7A.

FIG. 8A is a front view illustrating a two-rib-variable hip resurfacing implant with a fixed central stem, in accordance with another exemplary embodiment of the variable hip resurfacing implant of the present invention.

FIG. 8B is a cross-sectional left view of the two-rib-variable hip resurfacing implant shown in FIG. 8A, as indicated by line 8B-8B in FIG. 8A.

FIG. 8C is a right view of the two-rib-variable hip resurfacing implant shown in FIG. 8A.

FIG. 8D is a cross-sectional front view of the two-rib-variable hip resurfacing implant shown in FIG. 8A, as indicated by line 8D-8D in FIG. 8C.

FIG. 8E is a bottom view of the two-rib-variable hip resurfacing implant shown in FIG. 8A.

FIG. 8F is a bottom perspective view of the two-rib-variable hip resurfacing implant shown in FIG. 8A.

FIG. 8G is an enlarged bottom perspective view of the two-rib-variable hip resurfacing implant shown in FIG. 8A in detail, as indicated by the dashed circle around FIG. 8F.

### DETAILED DESCRIPTION

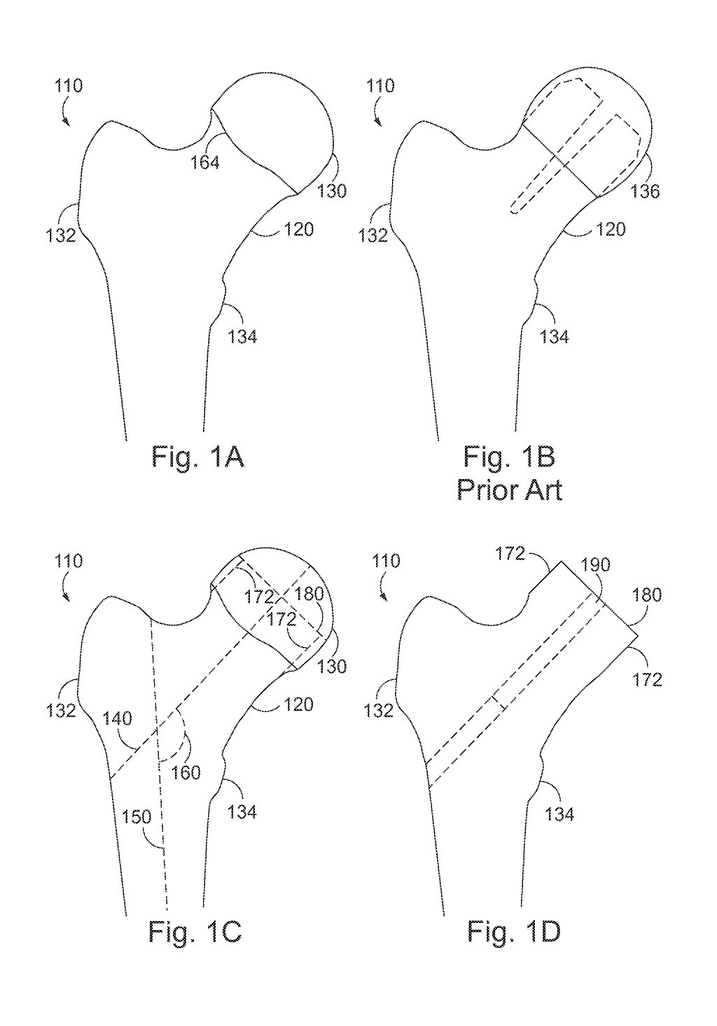

FIG. 1A is a front view from anterior of a human proximal femur 110 including femoral neck 120, femoral head 130, greater trochanter 132, lesser trochanter 134 and cartilage border, articular rim 164. FIG. 1B is a front view illustrating a proximal femur 110 following bone cuts and implantation of a hip resurfacing femoral implant 136, according to previous devices such as the Birmingham Hip Resurfacing implant. The dashed line in FIG. 1B illustrates the required bone cuts following cylindrical reaming and osteotomies, for hip resurfacing femoral implants according to previous devices. Such a hip resurfacing femoral implant, as illustrated, has a solid stem without internal threading. Bone preparations for implantation of this hip resurfacing implant include cylindrical reaming of the femoral head 130. This implant lacks threaded holes along its undersurface to accommodate optional threaded modular attachments, thus limiting implantation options.

FIG. 1C is a front view of a proximal femur 110 illustrating the femoral neck 120, femoral head 130 and reference lines for neck-shaft angle 160, which most commonly lies between 120° to 140°, and is calculated based on the angle created by the intersection of the femoral neck axis 140 relative to the femoral shaft axis 150.

In preparation for the variable hip resurfacing implant 210 according to the present invention, associated bone cuts are to be performed. In accordance with an exemplary embodiment of the present invention, for implantation of the variable hip resurfacing implant 210, these bone cuts include a femoral head cylindrical osteotomy 172 and proximal osteotomy 180. The proximal osteotomy 180 is performed at a femoral neck-osteotomy angle of approximately 90° in reference to the femoral neck axis 140. Alternately, a proximal osteotomy 180 is performed without the femoral head cylindrical osteotomy 172 which retains cortical bone of the femoral head 130, laterally. The central bone channel 190 is also drilled during bone preparations as detailed below. FIG. 1D is a front view of a proximal femur 110 following osteotomies and central bone channel drilling. The central bone channel can be drilled to a specific depth based on a selected exemplary embodiment, as illustrated with dashed lines in FIG. 1D.

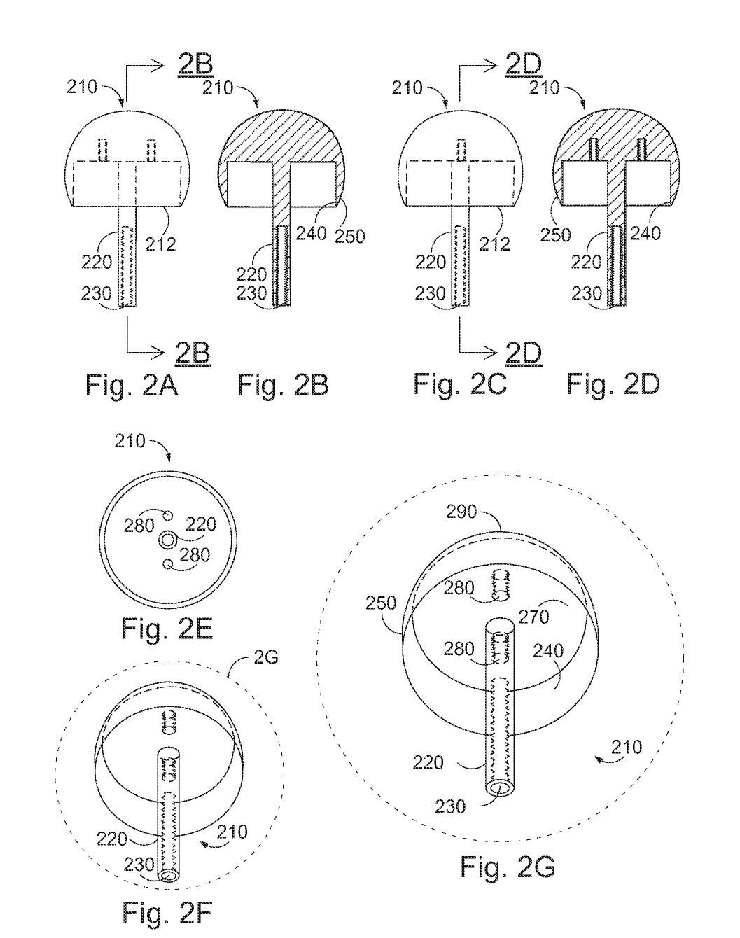

FIG. 2A is a front view illustrating various aspects of a variable hip resurfacing implant 210, in accordance with an exemplary embodiment of the implant according to the present invention. The variable hip resurfacing implant allows for modular attachment of multiple threaded accessories to increase fixation in the setting of bone deficiencies such as avascular necrosis. This implant 210 includes an upper, cap portion 212 and a lower, central stem portion 220. The non-removable, fixed, central stem is attached to the variable hip resurfacing implant and centrally joined to the undersurface of the cap portion 212.

The variable hip resurfacing implant may be produced with central stems of varying lengths to accommodate anatomic variation, as needed. FIG. 2B is a cross-sectional left view of the variable hip resurfacing implant as indicated by line 2B-2B in FIG. 2A. The central stem 220 also has an internally threaded, central stem threaded hole 230 at the lower aspect of its stem. FIG. 2C is a right view of the variable hip resurfacing implant. FIG. 2D is a cross-sectional front view of the variable hip resurfacing implant, as indicated by line 2D-2D in FIG. 2C.

FIG. 2E is a bottom view of the variable hip resurfacing implant 210 further illustrating the undersurface of the implant. FIG. 2F is a bottom perspective view of the variable hip resurfacing implant further showing its undersurfaces. FIG. 2G is an enlarged bottom perspective view of the variable hip resurfacing implant in detail, as indicated by the dashed circle around FIG. 2F. Following preparation of the proximal femur 110, the central stem 220 of the implant 210 inserts into the central bone channel 190. The inner rim 240 can provide peripheral stability for the variable hip resurfacing implant, further constraining its movement. The implant can be produced with variable heights with respect to the height of the inner planar surface 270 relative to the base of the cap portion 212 in 1 mm increments. This allows for variable degree of femoral head bone resection. This is beneficial when increased or decreased bone resection is necessary to address varying degrees of avascular necrosis within the femoral head 130.

The outer, bearing surface 290 of the cap portion 212 of the implant 210 extends just beyond a hemispherical shape, as illustrated. This area beyond equator is comprised of the inner rim 240 and outer rim 250. The inner rim surrounds the cancellous bone 330 of the femoral head 130 following the osteotomies.

The undersurface of the cap portion 212 has two internally threaded, implant threaded holes 280. In applications utilizing the implant threaded holes for modular threaded attachments, typically one implant threaded hole is oriented superiorly relative to the most proximal aspect of the femoral neck 120 and the other oriented inferiorly relative to the most distal aspect of the femoral neck 120. These implant threaded holes are located along the undersurface of the upper aspect, cap portion 212, as illustrated. The variable hip resurfacing implant allows multiple modular fixation options using its multiple modular attachment points which include the central stem threaded hole 230 and the two implant threaded holes 280. In the setting of decreased bone quality, additional modular threaded attachments can be used to supplement implant fixation.

The outer surface of the cap portion 212 has a bearing surface 290. The variable hip resurfacing implant 210 can be produced with varying cap portion diameters to accommodate anatomical differences of femoral head sizes. Various size options for the variable hip resurfacing implant include cap portions typically sized from 38 mm to 58 mm in 1 mm increments, with additional sizes for custom applications as needed. The cap portion can be produced in multiple diameters by decreasing or increasing, respectively, the distance between the inner rim 240 and outer rim 250. This is accomplished by making the entire cap portion larger while retaining the substantially hemispherical shape of its bearing surface, as the entire outer surface is enlarged accordingly.

Along the bottom edge of the bearing surface is the outer rim 250. A porous, textured, granular and/or beaded surface may be used on all undersurfaces and on the central stem of the variable hip resurfacing implant 210 for promotion of bony ingrowth at the interface of the implant and bone, as is known in the art. This porous surface is present on the implant at the interface of the implant and femoral bone. The porous surface provides additional construct strength over time with development of bony ingrowth. The undersurfaces and central stem may also include a hydroxy apatite coating to further promote bone ingrowth. The inner planar surface 270 of the variable hip resurfacing implant seats to the cylindrical planar surface of the remaining cancellous, trabecular, spongy bone of the femoral head following osteotomies, specifically the proximal osteotomy 180.

The variable hip resurfacing implant 210 including its fixed stem may be manufactured using a high carbon cobalt chrome (CoCr) alloy. The variable hip resurfacing implant may also be made of an alternate metal or metal alloy including stainless steel, titanium, zirconium, ceramic, ceramic coated metal, oxidized metal, or another suitable material. The central stem 220, and cap portion 212 of the implant may be manufactured as cast or using additional standard techniques including injection molding or forging. The central stem 220 and cap portion 212 of the implant may be manufactured separately and welded together or otherwise joined. In that case, a central hole can be present in the femoral head portion of the implant where the central stem may be inserted and joined using welding processes, or welding processes in addition to use of threading or other standard methods of joining metal, as is known in the art.

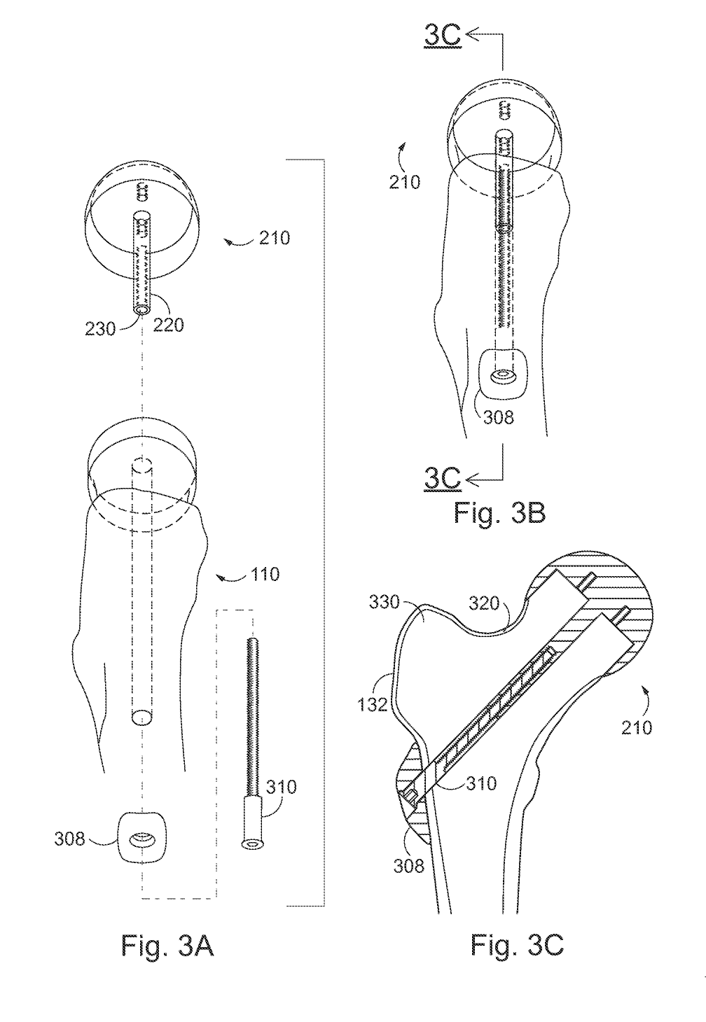

FIG. 3A is an exploded bottom perspective view illustrating placement of the variable hip resurfacing implant 210, one-hole plate 308, and central screw 310 into the prepared proximal femur 110, in accordance with one exemplary modular configuration of the present invention. The femoral head osteotomies and drilling of the central bone channel through the lateral femoral cortex are completed prior to implantation.

The central stem 220 of the variable hip resurfacing implant 210 is inserted into the central bone channel and the implant is seated. The one-hole plate 308 is placed onto the lateral femoral cortex and a central screw 310 is inserted through the plate 308 into the central bone channel and twisted, engaging the central stem threaded hole 230, with the plate 308 oriented along the femur as illustrated. Central screw tightening is completed once the variable hip resurfacing implant 210 is fully seated, and the central screw 310 is tightly secured into the central stem threaded hole 230.

FIG. 3B is a bottom perspective view following placement of the variable hip resurfacing implant 210, one-hole plate and central screw. FIG. 3C is a cross-sectional right view, as indicated by line 3C-3C in FIG. 3B, further illustrating the construct. Final seating is illustrated of the variable hip resurfacing implant 210 with the central screw 310 securely tightened. The one-hole plate 308 is positioned below the greater trochanter 132. The implant 210 is mated to both the cortical bone 320 and the cancellous bone 330.

Implantation of this exemplary modular configuration of the hip resurfacing system may be accomplished through the following steps. A posterior surgical approach to the hip joint is performed and the lateral cortex of the femur is exposed for access. The soft tissues are elevated just distal to the greater trochanter 132 where the plate 308 can be seated onto the lateral cortex. The hip is surgically dislocated posteriorly following a standard capsulotomy and careful capsular release in standard fashion for hip resurfacing arthroplasty. Femoral head cylindrical reaming is performed using a cylindrical reamer and the proximal femoral head osteotomy is performed using a saw guide and surgical oscillating saw and surgical caliper as needed, in standard fashion for hip resurfacing arthroplasty. The central bone channel 190 is also drilled parallel to the femoral neck angle fully through the lateral cortex in this modular configuration. These bone preparations provide mating surfaces along the femoral head 130 and femoral neck 120 and a shape fit for the seated implant. Preparation for placement of a standard acetabular component, which is proportionally sized, is undertaken and the acetabular component is implanted, as routinely performed for hip resurfacing arthroplasty.

The variable hip resurfacing implant 210 is fully inserted into the central bone channel 190 with an implant threaded hole oriented towards the remaining superior anatomic cortex of the femoral head 130. The one-hole plate 308 is placed on the lateral cortex and the central screw 310 is inserted through the plate and twisted, engaging the central stem threaded hole 230. The variable hip resurfacing implant is fully seated once its inner planar surfaces are mated to the osteotomized planar surface of the femoral head 130 and the central screw is tightly secured into the central stem threaded hole 230, as illustrated in FIG. 3C.

The resurfaced hip is relocated into the acetabular component and the hip capsule closed, in standard fashion for hip resurfacing, and the deep and superficial soft tissues are then sutured. This exemplary modular configuration is beneficial as it can be implanted following cylindrical reaming, as common in hip resurfacing, and additional bone resection and can be implanted in the setting of good bone quality in the superior and inferior femoral head, and the plate and central screw provide strong fixation, with the plate on the lateral cortex acting as a further buttress.

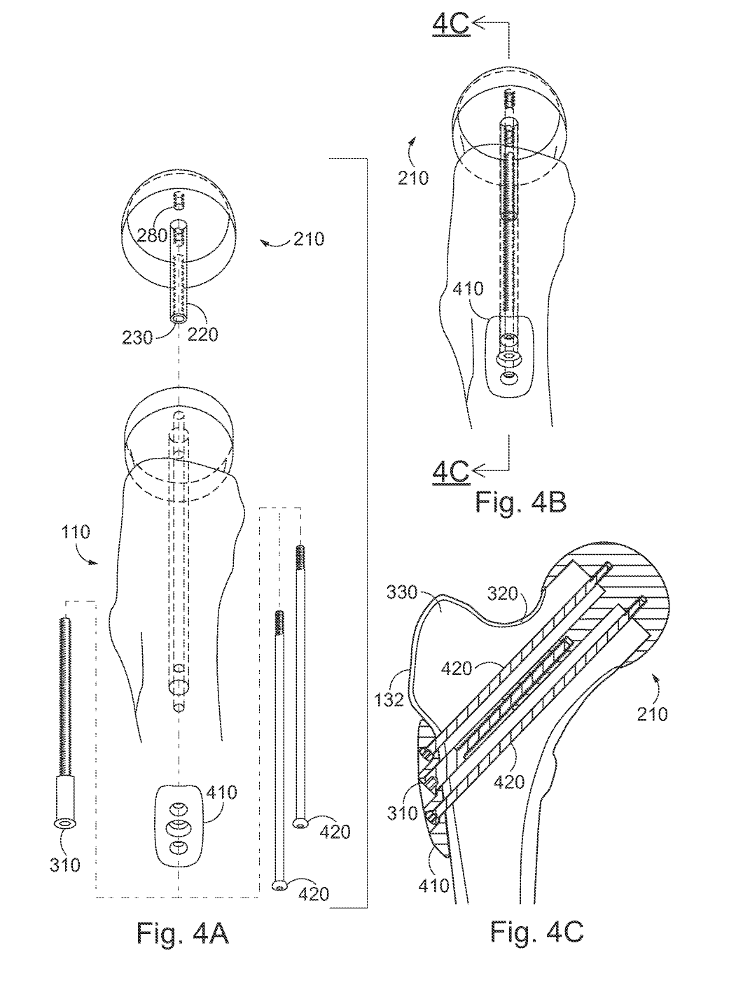

The exemplary modular configuration illustrated in FIGS. 4A to 4C is the same as that illustrated in FIGS. 3A to 3C with a few differences, which are described below. FIG. 4A is an exploded bottom perspective view illustrating placement of the variable hip resurfacing implant 210, three-hole plate 410, two outer screws 420, and a central screw 310, into the prepared proximal femur 110, in accordance with another exemplary modular configuration. FIG. 4B is a bottom perspective view, and FIG. 4C is a cross-sectional right view, as indicated by line 4C-4C in FIG. 4B, further illustrating the final construct.

The steps required for implantation are the same as those described with reference to FIGS. 3A to 3C with the following few differences. Two additional holes are drilled in order to accommodate two outer screws using a surgical caliper. Provisional guide pins are placed first, aided with fluoroscopic image guidance. These drilled holes align with the implant threaded holes and are parallel to the central bone channel. Further, the holes are drilled with a long standard surgical cannulated drill bit attached to a surgical drill, and are drilled completely through the lateral cortex of the femur to allow full insertion of the two outer screws.

The variable hip resurfacing implant 210 is inserted into the central bone channel with an implant threaded hole oriented toward the superior most aspect of the remaining femoral head cortex and aligned with the implant threaded hole 280. A three-hole plate is placed on the lateral cortex and two outer screws are inserted through the top and bottom holes of the plate. The outer screws are partially tightened after engaging the threads of the implant threaded holes. Then the central screw is inserted engaging the central stem threaded hole 230 and is tightened. Full tightening of the outer screws is then completed.

The outer screws provide further resistance to rotational forces during central screw tightening. The outer screws also provide additional fixation in the setting of decreased bone quality. The variable hip resurfacing implant 210 is fully seated, with the central screw tightly secured into the central stem threaded hole. The outer screws are also tightly secured.

The resurfaced hip is relocated into the acetabular component and the hip capsule closed, in standard fashion for hip resurfacing, and the deep and superficial soft tissues are then sutured. This exemplary modular configuration is beneficial as it can be implanted following cylindrical reaming, as common in hip resurfacing, and additional bone resection and can be implanted in the setting of decreased bone quality in the superior and inferior femoral head 130 and neck 120 as the outer screws in combination with the three-hole plate and central screw provide strong fixation, and the plate on the lateral cortex acts as a further buttress.

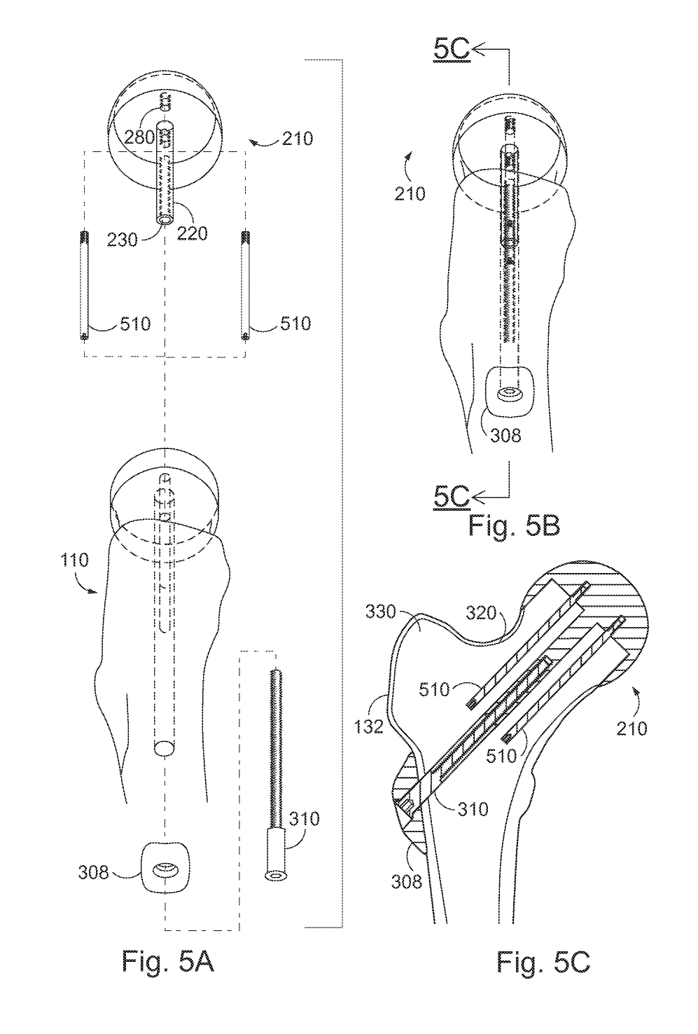

The exemplary modular configuration illustrated in FIGS. 5A to 5C is the same as that illustrated in FIGS. 4A to 4C with a few differences described below. FIG. 4A is an exploded bottom perspective view illustrating placement of the variable hip resurfacing implant 210, two outer pegs 510, one-hole plate 308, and a central screw 310 into the prepared proximal femur 110, in accordance with another exemplary modular configuration. FIG. 5B is a bottom perspective view, and FIG. 5C is a cross-sectional right view, as indicated by line 5C-5C in FIG. 5B, further illustrating the final construct.

The steps for implantation are the same as those described with reference to FIGS. 4A to 4C with a few differences described below. The upper and lower outer holes are drilled to accommodate two outer pegs 510, using guide pins, fluoroscopic guidance and cannulated drill bits, above and below the central bone channel and are drilled to match the length of the selected outer pegs 510. The selected outer pegs 510 are threaded into the top and bottom implant threaded holes and tightly secured. The variable hip resurfacing implant 210 is inserted into the prepared proximal femur 110, sliding the central stem with attached outer pegs 510 into the corresponding holes in the femoral head 130, as configured.

The variable hip resurfacing implant 210 is seated. A one-hole plate is placed on the lateral cortex. Then the central screw is inserted through the plate into the central bone channel and twisted, engaging the central stem threaded hole 230. The variable hip resurfacing implant is fully seated once its inner planar surfaces are mated to the osteotomized planar surface of the femoral head 130 and the central screw is tightly secured into the central stem threaded hole. Rotational forces during extended use of the implant are further also resisted by the outer pegs 510.

The hip is relocated into the acetabular component and the hip capsule is closed in standard fashion with sutures and the deep and superficial soft tissues are then sutured. The upper and lower outer pegs 510 attached to the variable hip resurfacing implant 210 in combination with the one-hole plate 308 and central screw are beneficial as this construct may be implanted following cylindrical reaming, as common in hip resurfacing, and additional bone resection and provides strong fixation setting of compromised bone quality in the femoral head and good bone quality in the femoral neck 120, and the plate on the lateral cortex acts as a further buttress.

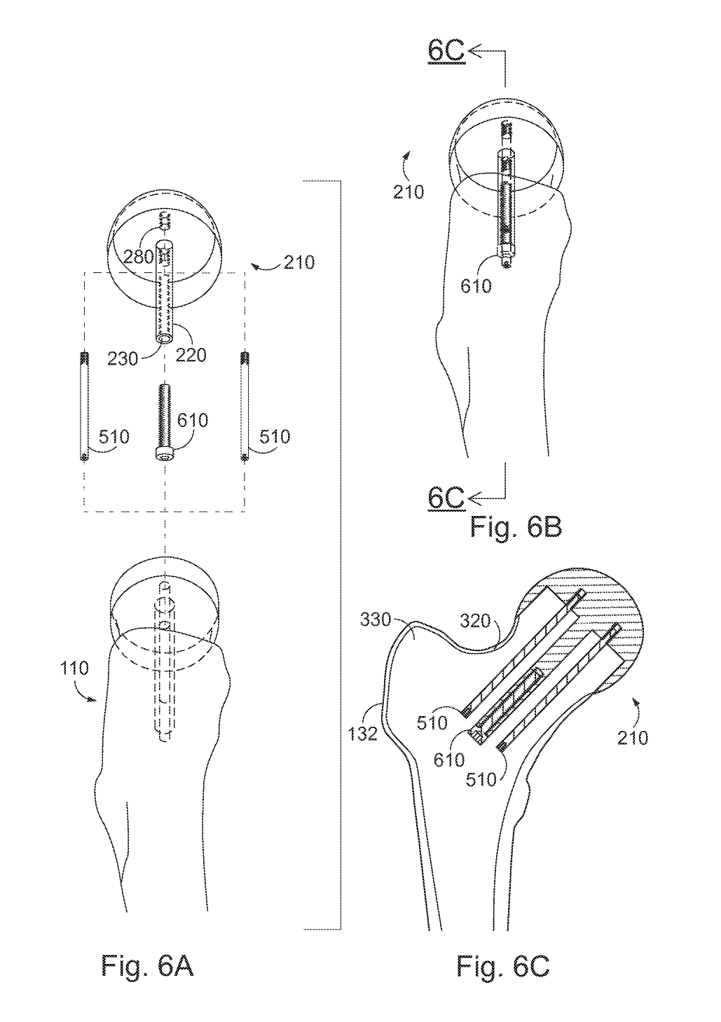

The exemplary modular configuration illustrated in FIGS. 6A to 6C is the same as that illustrated in FIGS. 5A to 5C with a few differences described below. FIG. 6A is an exploded bottom perspective view illustrating placement of the variable hip resurfacing implant 210, two outer pegs 510, and a threaded central cap 610 into the prepared proximal femur 110, in accordance with another exemplary modular configuration. FIG. 6B is a bottom perspective view, and FIG. 6C is a cross-sectional right view, as indicated by line 6C-6C in FIG. 6B, further illustrating the final construct.

The steps for implantation are the same as those described with reference to FIGS. 5A to 5C with a few differences described below. The lateral cortex of the femur is not prepared for plate placement, as a plate is not used in this exemplary modular configuration.

The central bone channel is drilled to the depth required for final seating of the variable hip resurfacing implant 210 with the attached threaded central cap, without reaching the lateral cortex. The depth of the central bone channel does not need to extend past the depth required for final seating in this modular configuration. A standard surgical drill depth gauge can be used to further facilitate this step. The upper and lower outer holes are drilled to accommodate two outer pegs, as described in FIG. 5A to 5C. The selected outer pegs are tightly secured into the implant threaded holes. The threaded central cap is tightly secured into the central stem threaded hole 230. Bone cement is placed along the cancellous bone 330 at the proximal osteotomy to mate with the implant inner planar surface. The variable hip resurfacing implant is inserted, sliding the central stem and attached outer pegs into the corresponding drilled holes, as configured. The variable hip resurfacing implant is inserted, and pressed into place until fully seated. The implant is firmly seated into place as illustrated.

Rotational forces during extended use of the implant are further also resisted by the outer pegs. The variable hip resurfacing implant 210 is fully seated once its inner planar surface is mated to the osteotomized planar surface of the femoral head 130. The resurfaced hip is relocated into the acetabular component and the hip capsule is closed in standard fashion with sutures and the deep and superficial soft tissues are then sutured. The benefits of this construct are that it can be implanted following cylindrical reaming, as common in hip resurfacing, and additional bone resection and the upper and lower outer pegs attached to the variable hip resurfacing implant in combination with the threaded central cap attached to the central stem in this cemented application provides strong fixation in a wide range of bone quality in the femoral head 130 and neck 120.

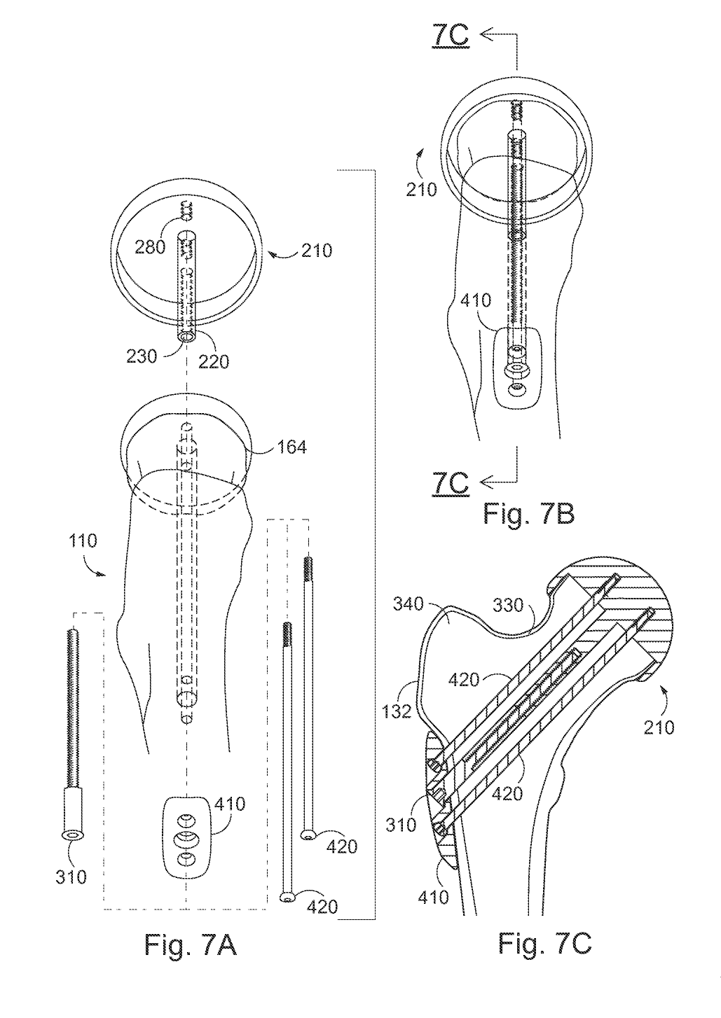

The exemplary modular configuration illustrated in FIGS. 7A to 7C is the same as that illustrated in FIGS. 4A to 4C with a few differences, which are described below. FIG. 7A is an exploded bottom perspective view illustrating placement of the variable hip resurfacing implant 210, three-hole plate 410, two outer screws 420, and a central screw 310, into the prepared proximal femur 110, in accordance with another exemplary modular configuration. FIG. 7B is a bottom perspective view, and FIG. 7C is a cross-sectional right view, as indicated by line 7C-7C in FIG. 7B, further illustrating the final construct.

The steps required for implantation are the same as those described with reference to FIGS. 4A to 4C with the following few differences. A proximal femoral head osteotomy has been performed above the cartilage border, articular rim 164, but without a femoral head cylindrical osteotomy. This retains a collar of femoral head cortical bone remaining above the cartilage border, articular rim 164. This cortical collar can provide additional implant support as it is comprised of dense bone. The proximal osteotomy can be aided through an alignment guide and surgical caliper, as known in the art. After drilling the central bone channel and two additional holes to accommodate the outer screws, the variable hip resurfacing implant 210 is inserted into the central bone channel with an implant threaded hole oriented toward the superior most aspect of the remaining femoral head cortex and aligned with the implant threaded hole 280. A three-hole plate is placed on the lateral cortex and two outer screws are inserted through the top and bottom holes of the plate. The outer screws are partially tightened after engaging the threads of the implant threaded holes. Then the central screw is inserted engaging the central stem threaded hole 230 and is tightened. Full tightening of the outer screws is then completed.

The outer screws provide further resistance to rotational forces during central screw tightening. The outer screws also provide additional fixation in the setting of decreased bone quality. The implant also seats along the cortical collar of bone retained in this embodiment which provides additional implant support. The variable hip resurfacing implant 210 is fully seated, with the central screw tightly secured into the central stem threaded hole. The outer screws are also tightly secured.

The resurfaced hip is relocated into the acetabular component and the hip capsule closed, in standard fashion for hip resurfacing, and the deep and superficial soft tissues are then sutured. This exemplary modular configuration is beneficial as it can be implanted following cylindrical reaming, as common in hip resurfacing, and additional bone resection and can be implanted in the setting of decreased bone quality in the superior and inferior femoral head 130 and neck 120 as the outer screws in combination with the three-hole plate and central screw provide strong fixation, and the plate on the lateral cortex acts as a further buttress.

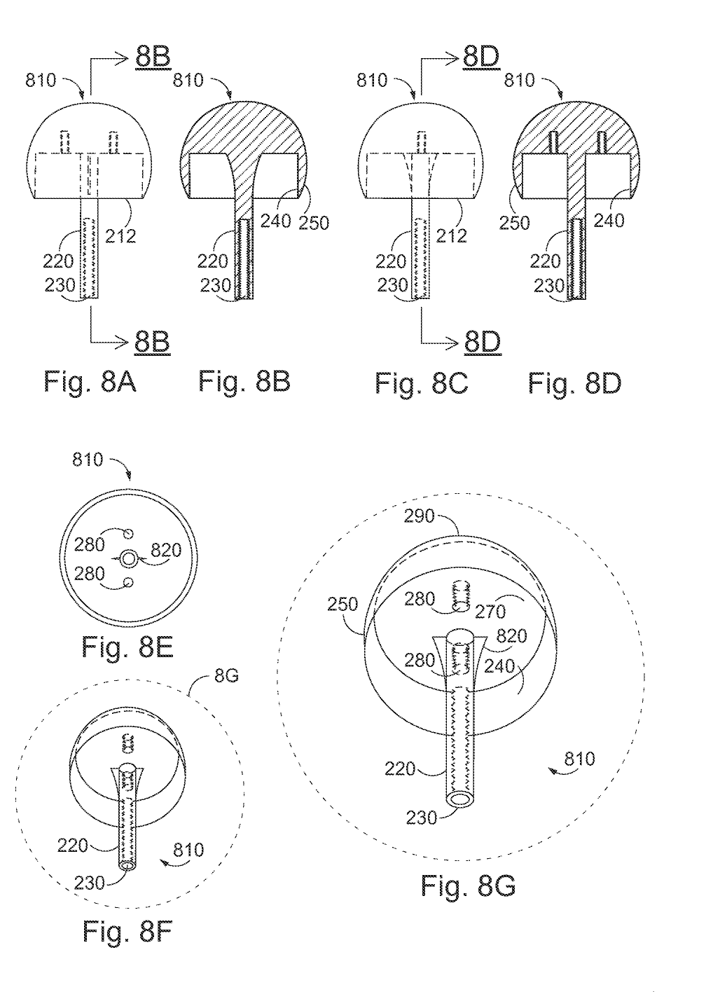

FIG. 8A is a front view and FIG. 8B is a cross-sectional left view, as indicated by line 8B-8B in FIG. 8A, both illustrating various aspects of a two-rib variable hip resurfacing implant 810, in accordance with another exemplary embodiment of the variable hip resurfacing implant according to the present invention. FIG. 8C is a right view of the two-rib-variable hip resurfacing implant and FIG. 8D is a cross-sectional front view of the two-rib-variable hip resurfacing implant, as indicated by line 8D-8D in FIG. 8C. FIG. 8E is a bottom view of the two-rib-variable hip resurfacing implant further showing its undersurface. The two-rib implant is the same as the implant described in FIGS. 2A to 2G with the following differences. In the present, two-rib exemplary embodiment, the central stem has two equally spaced ribs 820, circumferentially joined to the top of the central stem, joined to and extending from the undersurface of the cap portion partially downward along the central stem. The ribs are joined to the flat, inner planar surface 270 of the implant and are also joined to the central stem. They are located at opposite sides of the central stem, at 180 degrees from each other, as illustrated. The ribs extend from the inner planar undersurface of the implant down the central shaft to the level of the outer rim, but may vary in alternate embodiments. The ribs have sharp outer edges to ease implantation. The implant is designed to be inserted into the central bone channel with one of the two implant threaded holes 280 oriented superiorly relative to the femoral neck cortex. The ribs in this exemplary embodiment provide support for the implant in the axial plane, improve stress transmission from the femoral head portion of implant to the central stem, and provide further rotational control during implantation and extended use. A porous surface may be used on the implant undersurface in the same manner as the exemplary embodiment described in FIGS. 2A to 2G.

FIG. 8F is a bottom perspective view of the two-rib-variable hip resurfacing implant and FIG. 8G is an enlarged bottom perspective view of the two-rib-variable hip resurfacing implant in detail, as indicated by a dashed circle around FIG. 8F. The two-rib implant allows for the modular fixation options illustrated in FIGS. 3 to 7, the choice of which are based on individual characteristics of the underlying bone quality and severity of degenerative hip joint disease present. The two added ribs in this exemplary embodiment along the upper aspect of the central stem provide additional rotational resistance during implantation and extended use.

The variable hip resurfacing implant 210 of the present invention provides means for both un-cemented and cemented applications and options for multiple modular threaded attachments. Additionally, in multiple exemplary embodiments, additional construct support is provided, including fixation at the lateral cortex of the proximal femur 110 with placement of the central screw fitted through a plate. Additional modular options in this implant include use of outer screws, and outer pegs. In various exemplary embodiments, threaded attachments provide additional construct support for enhanced scalability and resistance to rotation when needed, including for example, outer screws and outer pegs.

While the above description contains much specificity, this should not be construed as limitations on the scope, but rather an exemplification of one or more exemplary embodiments as detailed. Multiple additional modular configurations are possible using the variable hip resurfacing system. For example, another modular configuration can be employed including the variable hip resurfacing implant 210, a single outer peg above or below the stem, a one-hole plate, and a central screw.

Furthermore, alternate embodiments of the variable hip resurfacing implant 210 can include an underside of the cap portion with a concave surface configured to match a convex shape following a cylindrical reaming of the femoral head 130 with a cylindrical reamer which produces a femoral head shape with a corresponding convex proximal aspect. The illustrated femoral head osteotomy in FIG. 1D and configuration of the illustrated embodiments of the variable hip resurfacing implant in FIGS. 5A to 5C, are presently preferred. An alternative osteotomy can be used for implantation through a single transverse osteotomy at an approximate 90° angle to the femoral neck angle, as demonstrated in FIG. 7A to 7C. This allows for an alternate implantation of a proportionality-sized variable hip resurfacing implant whereby the implant seats along the transverse osteotomy at its inner planar surface and along the cortical bone of the femoral head 130 at its inner rim. Another possible implant alteration is that the inner edges of the variable hip resurfacing implant can be modified so that the transition between the inner rim to inner planar surface is filleted or chamfered instead of its present 90° angle configuration. The present 90° angle configuration is preferred as it allows preservation of more cancellous bone 330 within the femoral head 130.

Additional alternate embodiments and ramifications are possible of the variable hip resurfacing implant 210 according to the present invention. The central stem can be modified so that it includes a tapered shape with a wider diameter at the upper aspect transitioning to a narrower diameter along its base. The central stem may also include a fluted surface along the perimeter to further increase rotational stability. The central stem may also be filleted or chamfered at the area it meets the inner planar surface.

The two-rib variable hip resurfacing implant 810 may be modified by joining additional ribs along the upper perimeter of the central stem. For example, a three-, four- or five-rib version of the variable hip resurfacing implant can be created in order to provide additional rotational stability. These ribs would be joined to both the central stem and undersurface of the implant as in the two-rib implant. Or a single-rib variable hip resurfacing implant can be produced which would ease implantation while still providing rotational stability through inclusion of a rib along the upper aspect of the central stem.

The central stem threaded hole may be omitted although its presence is preferred for providing further modular capabilities. The implant threaded holes may be moved on alternate embodiments to other locations on the implant undersurface, or additional implant threaded holes may be added; corresponding additions may be made to the outer hole drill guide. One or both of the implant threaded holes may also be omitted in alternate embodiments; however, they are desirable as they provide for increased modularity and stability when needed. For example, in the setting of poorer bone quality, the added construct rigidity afforded by addition of outer screws and/or outer pegs, may be especially beneficial and can help further increase durability. Additional embodiments may also include a version of the variable hip resurfacing implant with an inner rim that is taller or shorter with corresponding changes made to the bone cuts. Another embodiment of the implant can be a version with a removable central stem by adding both threading to the top of the stem and a central threaded hole along the inner planar surface to secure the removable central stem. A further additional embodiment of the implant may include a version without a central stem. Additionally, the central screw may be used to directly engage the cap portion of the modular stemmed implant at a central threaded hole along the base of the cap portion, thereby obviating the need for the removable central stem. Another additional embodiment of the variable hip resurfacing implant 210 has a central stem modified to have external exposed threads, and corresponding modifications made to its associated modular attachments including the central screw and central cap having internal threads. This would essentially change the central stem to male, external threads, and the central screw and central cap to female, internal threads.

Another alternate embodiment of the variable hip resurfacing implant 210 may include wider and longer ribs on the ribbed versions of the central stem, and also a one-, three- or more rib version can be produced. Additionally, with respect to the modular stemmed embodiment of the implant according to the present invention, a one-, two-, three-, four- or more rib version of the stem can be produced. Additionally, the plates may be modified to accommodate additional modular configurations. Specifically, a two-hole plate can be manufactured to accommodate the central screw with use of an outer screw or cortical screw below it. Additional plates and screws may also be modified to allow the screw threading to engage threading within the hole in the plate, as is well known in the art.

The variable hip resurfacing implant 210, screws, pegs, threaded central cap, and plates may each be made of metal or metal alloy including high carbon cobalt chrome (CoCr) alloy, stainless steel, titanium or aluminum, ceramic, ceramic coated metal or another suitable material, or combination of suitable materials with or without bearing surface coatings. They may be manufactured as cast or using additional standard techniques including injection molding, forging, bending during the machining process, and can be manufactured in segments and welded or otherwise joined, e.g., with an inference fit.

Although the present invention has been described with reference to particular examples and exemplary embodiments, it should be understood that the foregoing description is in no manner limiting. For example, although the present invention has been described with reference to particular materials, manufacturing methods and joining methods, it should be understood that other suitable materials may be employed, such as, for example, ceramics or plastics through solids, composites, and/or the addition of bearing surface coatings or substrates, or other manufacturing methods such as, for example, injection molding, laser cutting or alternate machining methods, and/or other joining methods such as, for example, friction fitting may be encompassed by the present invention. Moreover, the features described herein may be used in any combination.

What is claimed is:

1. A variable hip resurfacing implant, comprising:
a cap portion having an inner rim disposed along a periphery of an underside of the cap portion, wherein the inner rim has a non-stepped profile; and
a central stem disposed at the underside of the cap portion, wherein the central stem includes threading at a base of the central stem configured to accommodate threaded attachments.

2. The variable hip resurfacing implant according to claim 1, wherein the inner rim of the cap portion is configured such that it does not surround a femoral head cortex remaining above an articular rim of a femoral head upon seating of the implant.

3. The variable hip resurfacing implant according to claim 1, wherein the inner rim of the cap portion is configured such that it surrounds a femoral head cortex remaining above an articular rim of a femoral head upon seating of the implant.

4. The variable hip resurfacing implant according to claim 1, wherein the underside of the cap portion includes at least one implant threaded hole located between the central stem and the inner rim of the cap portion configured to attach to the threaded attachments.

5. The variable hip resurfacing implant according to claim 1, wherein the underside of the cap portion includes a central threaded hole configured to attach to a modular central stem or a modular central screw.

6. The variable hip resurfacing implant according to claim 1, wherein the central stem is removable.

7. The variable hip resurfacing implant according to claim 1, wherein the threading at the base of the central stem includes internal threading.

8. The variable hip resurfacing implant according to claim 1, wherein the threading at the base of the central stem includes external threading.

9. The variable hip resurfacing implant according to claim 1, wherein the central stem includes at least one external rib extending at least partially downward from the underside of the cap portion.

10. The variable hip resurfacing implant according to claim 1, wherein the underside of the cap portion includes at least one of (i) at least one surface configured to match at least one surface of the femoral head, (ii) at least one porous surface configured to promote bony ingrowth, or (iii) at least one threaded hole configured for attachment of the threaded attachments.

11. The variable hip resurfacing implant according to claim 1, further comprising at least one of:
a central screw configured to extend from a lateral femoral shaft cortex of a proximal femur to the threading at the base of the central stem;
a central cap configured to attach to the threading at the base of the central stem;
at least one outer screw configured to extend from the lateral femoral shaft cortex of the proximal femur to the at least one threaded hole on the underside of the cap portion; or
at least one outer peg attached to the at least one threaded hole on the underside of the cap portion.

12. The variable hip resurfacing implant according to claim 1, wherein the variable hip resurfacing implant is configured to be implanted with cement.

13. The variable hip resurfacing implant according to claim 1, further comprising at least one of:
a plate situated at a lateral femoral shaft cortex of a proximal femur and having at least one hole through which at least one of (i) a central screw or (ii) at least one outer screw extends.

14. A method for preparing a femoral head, a femoral neck and a proximal femur for implantation of a variable hip resurfacing implant, comprising:
performing a cylindrical femoral head osteotomy,
drilling a central bone channel through the femoral head and the femoral neck at least partially towards a lateral femoral shaft cortex of the proximal femur.
implanting the variable hip resurfacing implant including a cap portion having an inner rim disposed along a periphery of an underside of the cap portion, wherein the inner rim has a non-stepped profile, and a central stem disposed at the underside of the cap portion.

15. The method according to claim 14, wherein, following the cylindrical femoral head osteotomy, the cap portion does not surround a periphery of a cortex of the femoral head remaining above an articular rim of the femoral head.

16. The method according to claim 14, wherein the implanting of the variable hip resurfacing implant includes attaching at least one of:
a central screw configured to extend from the lateral femoral shaft cortex of the proximal femur to the threading at the base of the central stem;
a central cap configured to attach to the threading at the base of the central stem;
at least one outer screw configured to extend from the lateral femoral shaft cortex of the proximal femur to the at least one threaded hole on the underside of the cap portion; or
at least one outer peg attached to the at least one threaded hole on the underside of the cap portion.

17. The method according to claim 16, wherein the implanting of the variable hip resurfacing implant further includes:
placing a plate at the lateral femoral shaft cortex of the proximal femur, the plate having at least one hole through which at least one of (i) the central screw or (ii) the at least one outer screw extends.

18. The method according to claim 14, wherein the central stem includes threading at a base of the central stem configured to accommodate threaded attachments.

19. The method according to claim 14, wherein the underside of the cap portion includes at least one implant threaded hole located between the central stem and the inner rim of the cap portion configured to attach to threaded attachments.

20. A method for preparing a femoral head, a femoral neck and a proximal femur for implantation of a variable hip resurfacing implant, comprising:
performing a proximal femoral head osteotomy without a cylindrical femoral head osteotomy,
drilling a central bone channel through the femoral head and the femoral neck at least partially towards a lateral femoral shaft cortex of the proximal femur.
implanting the variable hip resurfacing implant including a cap portion having an inner rim disposed along a periphery of an underside of the cap portion, wherein the inner rim has a non-stepped profile, and a central stem disposed at the underside of the cap portion.

21. The method according to claim 20, wherein, following the proximal femoral head osteotomy, the cap portion surrounds a periphery of a cortex of the femoral head remaining above an articular rim of the femoral head.

22. The method according to claim 20, wherein the implanting of the variable hip resurfacing implant includes attaching at least one of:
a central screw configured to extend from the lateral femoral shaft cortex of the proximal femur to the threading at the base of the central stem;
a central cap configured to attach to the threading at the base of the central stem;
at least one outer screw configured to extend from the lateral femoral shaft cortex of the proximal femur to the at least one threaded hole on the underside of the cap portion; or
at least one outer peg attached to the at least one threaded hole on the underside of the cap portion.

23. The method according to claim 20, wherein the implanting of the variable hip resurfacing implant further includes:
placing a plate at the lateral femoral shaft cortex of the proximal femur, the plate having at least one hole through which at least one of (i) the central screw or (ii) the at least one outer screw extends.

24. The method according to claim 20, wherein the central stem includes threading at a base of the central stem configured to accommodate threaded attachments.

25. The method according to claim 20, wherein the underside of the cap portion includes at least one implant threaded hole located between the central stem and a central rim of the cap portion configured to attach to threaded attachments.

26. A variable hip resurfacing implant, comprising:
a cap portion having an inner rim disposed along a periphery of an underside of the cap portion, wherein the inner rim has a non-stepped profile; and
wherein the underside of the cap portion includes at least one implant threaded hole located between a central stem and the inner rim of the cap portion configured for attachment of threaded attachments.

27. The variable hip resurfacing implant according to claim 26, wherein the central stem includes internal threading at a base of the central stem.

28. The variable hip resurfacing implant according to claim 26, wherein the central stem includes external threading at a base of the central stem.

29. The variable hip resurfacing implant according to claim 26, wherein the central stem does not include threading at a base of the central stem.
